(19) United States
(12) Patent Application Publication
(10) Pub. No.: US 2022/0093231 A1
(43) Pub. Date: Mar. 24, 2022

(54) ESTIMATING GENERATION RATE OF SUBSTANCE IN DIALYSIS PATIENTS

(71) Applicant: GAMBRO LUNDIA AB, Lund (SE)

(72) Inventor: Jan STERNBY, Lund (SE)

(21) Appl. No.: 17/424,406

(22) PCT Filed: Feb. 12, 2020
(86) PCT No.: PCT/EP2020/053563

§ 371 Date: Jul. 20, 2021

(30) Foreign Application Priority Data
Mar. 11, 2019 (SE) 1950303-6

(51) Int. Cl. G16H 20/40; G16H 10/40

(52) U.S. Cl. CPC G16H 20/40; G16H 10/40

(57) ABSTRACT

A computer system may implement a method of calculating the generation rate of a substance in a dialysis patient, e.g. urea, creatinine or beta-2-microglobulin, based on data for one or more treatment sessions of intermittent dialysis therapy. The computer system obtains a concentration value (Cs) for the substance in the blood of the dialysis patient at the start of a treatment session, and a standard Kt/V value (stdKt/V) for the substance over a predefined time period (t), which includes the one or more treatment sessions. The computer system then computes the generation rate (G; G/V) as a function of stdKt/V, the first blood concentration value (Cs) and the predefined time period (t).

### TECHNICAL FIELD

The present invention relates to techniques for estimating the generation rate of a substance, including but not limited to urea, in dialysis patients.

### BACKGROUND ART

Malnutrition is common in dialysis patients and is associated with a high mortality and morbidity. Malnutrition may be caused by underdialysis, inflammation or loss of appetite. The early signs of malnutrition are easy to miss in dialysis patients, particularly if there is no weight loss due to fluid accumulation.

The nutritional status of a patient may be assessed by measuring the generation rate of urea, which is a by-product of protein metabolism. Thus, the patient's protein intake is related to the appearance of urea in the spent dialysis fluid or ultrafiltrate that is produced during a dialysis session, and in the patient's urine if the patient has a residual renal function.

In continuous dialysis therapies, such as peritoneal dialysis and continuous hemodialysis, the urea generation rate is given by the product of the urea clearance and the urea concentration in the blood of the dialysis patient. Such calculation is trivial in continuous dialysis treatment since the blood urea concentration is constant over time and the urea clearance is obtainable.

However, in intermittent dialysis therapies, it is more difficult to obtain the urea generation rate for assessment of the nutritional status of the dialysis patient.

One known technique is to measure the amount of urea in spent dialysis fluid or ultrafiltrate, e.g. by an on-line urea sensor in the dialysis machine. In a steady state condition, the total amount of urea removed during dialysis sessions and sensed by the urea sensor is equal to the rate of generation of urea in the patient's body and may be used for assessing the nutritional status, e.g. by calculating the protein catabolic rate

(PCR) or the number of grams of urea generated per kilogram of body mass in the related time period. Such techniques are, e.g., disclosed in WO2011/147425, WO94/08641, US2014/0190886, WO94/09351, WO98/55116, and the article “On-line Urea Monitoring During Hemodialysis: A Review”, by Stiller et al., published in Saudi J Kidney Dis Transplant 12(3):364-374 (2001). As noted, these techniques presume a steady state condition and thereby require the amount of urea to be measured and aggregated over a plurality of treatment sessions, which may be impractical. Further, the need for one or more urea sensors will increase the cost of the dialysis machine. While there are commercially available dialysis machines with integrated on-line monitoring equipment, a majority of the dialysis machines presently in use lack such functionality. Retrofitting existing dialysis machines with on-line monitoring functionality is also too expensive to be a realistic option.

Another known technique, which is applicable to all intermittent dialysis therapies, is to take blood samples at the beginning and end of a dialysis session and at the beginning of the next dialysis session, determine blood urea concentrations in the three blood samples, and perform a 3-point urea kinetic modeling (UKM) based on the blood urea concentrations. UKM may be implemented to yield the relative urea generation rate, i.e. the urea generation rate in the dialysis patient in relation to the water volume of the dialysis patient. However, UKM involves iterative computations for solving coupled equations and is thus relatively complex. Further, 3-point UKM requires three blood samples to be collected in two consecutive dialysis sessions. Generally, cost and complexity increase with every blood sample that needs to be taken and analyzed by specialized equipment in a laboratory.

Presently, it is common practice at dialysis clinics to regularly perform blood tests of dialysis patients, e.g. once every month, to assess the blood concentration of albumin, urea, calcium, phosphate, etc. It is also common practice to periodically, normally once a month, assess the dialysis adequacy of intermittent dialysis therapies by sampling blood at the start and at the end of a dialysis session and comparing the levels of urea in the two blood samples, e.g. by calculating the urea reduction ratio (URR) or the Kt/V of the dialysis session (“session Kt/V”).

The urea generation rate is only indirectly related to the dietary protein intake (DPI) and misjudgment of the nutritional status may result if the dialysis patient is in negative nitrogen balance and relatively catabolized, which is quite possible for a malnourished patient. For example, the urea generation rate may be seemingly normal in a malnourished patient if urea is generated by muscle catabolism. Thus, the urea generation rate may need to be supplemented by other dietary assessment tools, including assessment of muscle mass. Creatinine is a well-known marker of muscle mass. Like urea, creatinine generation rate may be determined by analysis of three blood samples taken at two subsequent dialysis sessions and 3-point kinetic modeling.

The creatinine generation rate may also be used independently of the urea generation rate for assessing the physiological status of the patient. The generation rate of further substances in a dialysis patient may also be of interest for assessing the physiological status or for use in kinetic modeling. For example, amyloidosis is a known complication in dialysis patients in which an abnormal protein called amyloid builds up in the patient's tissues and organs. A major component of the amyloid is beta-2-microglobin (B2M). The generation rate of B2M in a dialysis patient may be relevant input for accurate assessment of the risk for future amyloidosis and for determining how to adjust the treatment plan accordingly.

### SUMMARY

It is an objective of the invention to at least partly overcome one or more limitations of the prior art.

A further objective is to provide an alternative technique for estimating the generation rate of a substance in a dialysis patient.

Another objective is to provide such a technique which is cost effective and may be implemented for all intermittent dialysis therapies.

Yet another objective is to provide such a technique which allows the generation rate to be determined from measurements in a single dialysis session. One or more of these objectives, as well as further objectives that may appear from the description below, are at least partly achieved by a method, a computer-readable medium and a computer device in accordance with the independent claims, embodiments thereof being defined by the dependent claims.

A first aspect of the invention is a method of calculating a generation rate of a substance in a dialysis patient. The method comprises: obtaining a first concentration value for the substance in the blood of the dialysis patient at the start of a treatment session of intermittent dialysis therapy; obtaining a standard Kt/V value for the substance over a predefined time period, which includes the treatment session; and calculating the generation rate of the substance in the dialysis patient as a function of the standard Kt/V value, the first concentration value and the predefined time period.

The first aspect is based on the insight that the generation rate of a substance may be derived by simple and straight-forward calculation by use of a parameter known as “standard Kt/V” in the art, commonly abbreviated “stdKt/V”. This parameter is a well-known and established measure of dialysis adequacy and has been developed to enable comparison of a broad spectrum of dialysis therapies, including intermittent hemodialysis therapies, continuous and intermittent ultrafiltration therapies, continuous and intermittent peritoneal dialysis, and continuous hemodialysis therapies for acute renal failure. Although the parameter is commonly derived for urea, it is generally applicable to any substance that is extracted from the blood of the dialysis patient in dialysis treatment. In accordance with its underlying definition, stdKt/V is given as G·t/(C̅s̅·V), where G is the generation rate of a substance in the dialysis patient, t is a predefined time period, C̅s̅ is the average predialysis concentration of the substance in the blood of the dialysis patient over the time period t, and V is the distribution volume in the dialysis patient of the substance. By insightful reasoning, the inventor has realized that if stdKt/V is known, it is possible to apply the underlying definition of stdKt/V to directly compute the generation rate G or the relative generation rate G/V based on the known value of stdKt/V, provided that the average predialysis concentration C̅s̅ is measured or estimated for the time period t.

A number of different computation algorithms have been developed that relate stdKt/V to known or measurable parameters of dialysis therapy. Generally, the existing computation algorithms for stdKt/V are given either as a function of the session Kt/V of the substance for the respective treatment session within the time period t, or as a function of the blood concentrations of the substance at the start and end of the respective treatment session within the time period t. The computation algorithms for stdKt/V further operate on the duration of the respective treatment session and the total fluid volume (if any) removed from the blood by dialysis therapy over the time period t. However, to the extent that the predefined time period includes more than one treatment session, there are computation algorithms that enable stdKt/V to be approximated based on measured data for a single treatment session, be it session Kt/V or blood concentrations at start and end of the treatment session. Generally, such an approximation introduces relatively small inaccuracies in the stdKt/V value. Thus, in accordance with some embodiments of the first aspect, a stdKt/V value is estimated at least based on measured data for one treatment session during the predefined time period and may, but need not, be based also on corresponding measured data for one or more further treatment sessions during the predefined time period. It should be understood that the settings of the dialysis therapy may differ between the treatment sessions during the predefined time period. Further, different types of intermittent dialysis therapy may be employed in different treatment sessions during the predefined time period, e.g. any combination of hemodialysis, hemodiafiltration, hemofiltration, ultrafiltration and peritoneal dialysis.

The above-mentioned blood concentrations may be obtained from blood samples taken in connection with the treatment session. The session Kt/V of the substance for the respective treatment session may be computed as a function of the blood concentrations, as known in the art, e.g. by formal kinetic modeling or by use of established equations for single-pool Kt/V, single-pool variable volume Kt/V, double-pool Kt/V, or equilibrated Kt/V. In addition to blood concentrations, such computations of the session Kt/V may operate on the volume of fluid removed from the blood during the treatment session, the body weight of the patient, and the effective dialysis time during the treatment session. Alternatively, the session Kt/V may be obtained by straight-forward calculation based on the clearance K of the substance, the effective dialysis time and the distribution volume V. There are established techniques for measuring or estimating the in-vivo clearance K of a substance for a treatment session. For example, the in-vivo clearance K may be determined by generating a short-term bolus of a parameter of the dialysis fluid entering the dialyzer and by measuring this parameter at least downstream of the dialyzer, e.g. as disclosed in U.S. Pat. Nos. 5,024,756, 5,100,554, EP0658352 and U.S. Pat. No. 6,702,774. There are commercially available devices that measure the in-vivo clearance in dialysis systems, e.g. DIASCAN from Gambro/Baxter, and Online Clearance Monitoring (OCM) from Fresenius. Depending on implementation, such a measurement device may output a clearance value K or a corresponding session Kt/V.

The first aspect provides a novel and alternative technique for estimating the generation rate of a substance in a dialysis patient. The first aspect may be implemented for any intermittent dialysis therapy and any combinations of such therapies. The first aspect may also provide an average generation rate for the predetermined time period t, rather than the more short-term generation value that is derived by the above-mentioned 3-point kinetic modeling. Such an average generation rate is inherently less sensitive to incidental short-term variations that may give a misleading picture of the physiological status of the patient. Also, the first aspect allows the generation rate to be determined from measurement data for a single treatment session, if both stdKt/V and the average predialysis concentration C̅s̅ are given by such measurement data. This will result in a cost-effective and time-efficient procedure. For example, the generation rate may be computed based on concentration values given by the blood samples that are taken anyway for regular assessment of the dialysis adequacy (cf. Background section) and/or based on a session Kt/V that may be obtained for a treatment session by use of commercially available and standard equipment.

In the following, various embodiments of the first aspect are defined. These embodiments provide at least some of the technical effects and advantages described in the foregoing, as well as additional technical effects and advantages as readily understood by the skilled person, e.g. in view of the following detailed description.

In one embodiment, the method further comprises: obtaining a session Kt/V value for the treatment session or a second concentration value for the substance in the blood of the dialysis patient at the end of the treatment session; obtaining a volume value representative of total fluid volume removed from the blood during the predefined time period; and obtaining a duration of the treatment session; wherein said obtaining the standard Kt/V value comprises computing the standard Kt/V value as a function of the volume value, the duration, and one of the session Kt/V value and the first and second concentration values.

In one embodiment, the substance is one of urea, creatinine and beta-2-microglobulin. In one embodiment, said calculating the generation rate comprises multiplying the standard Kt/V value, the reciprocal of the predefined time period, and an estimated concentration value, which is representative of an average predialysis concentration of the substance in the blood of the dialysis patient during the predefined time period.

In one embodiment, the method further comprises determining the estimated concentration value as a function of the first concentration value. In one example, the estimated concentration value is set in relation to the first concentration value. In another example, the estimated concentration value is computed as an average of the first concentration value and one or more further concentration values for the substance in the blood of the dialysis patient at the start of one or more further treatment sessions of intermittent dialysis therapy during the predefined time period.

In one embodiment, the predefined time period is selected so that the concentration of the substance in the blood of the dialysis patient is substantially equal at the start and end of the predefined time period.

In one embodiment, the predefined time period is a week. In one embodiment, the predefined time period includes one or more further treatment sessions, and the standard Kt/V value is estimated to include the one or more further treatment sessions.

In one embodiment, the standard Kt/V value is estimated in absence of concentration values for the substance in the blood of the dialysis patient during the one or more further treatment sessions and in absence of a Kt/V value for the one or more further treatment sessions. For example, the treatment session may be selected so that the first concentration value of the treatment session is closest to an average predialysis concentration of the substance in the blood of the dialysis patient during the predefined time period, compared to an expected concentration value for the substance in the blood of the dialysis patient at the start of the respective further treatment session.

In one embodiment, the method further comprises one or more of: displaying the generation rate, evaluating the generation rate for assessment of a physiological status of the dialysis patient, and displaying a parameter value representing the physiological status of the dialysis patient.

In one embodiment, the method is performed subsequent to said treatment session.

A second aspect of the invention is a computer-readable medium comprising computer instructions which, when executed by a processor, cause the processor to perform the method of the first aspect or any of its embodiments.

A third aspect is a computer system for calculating a generation rate of a substance in a dialysis patient. The computer system is configured to obtain a first concentration value for the substance in the blood of the dialysis patient at the start of a treatment session of an intermittent dialysis therapy, obtain a standard Kt/V value for the substance over a predefined time period, which includes the treatment session; and calculate the generation rate of the substance in the dialysis patient as a function of the standard Kt/V value, the first concentration value and the predefined time period.

Any one of the embodiments of the first aspect may be adapted and implemented as an embodiment of the third aspect.

Still other objectives, features, embodiments, aspects and advantages of the present invention may appear from the following detailed description, from the attached claims as well as from the drawings.

### BRIEF DESCRIPTION OF THE DRAWINGS

Embodiments of the invention will now be described in more detail with reference to the accompanying drawings.

FIG. 1 is a schematic overview of a system for assessment of the nutritional status of a dialysis patient.

FIG. 2 is an example graph of blood urea concentration of a dialysis patient that undergoes three dialysis treatment sessions during the course of a week.

FIG. 3 is a flow chart of a method of calculating the generation rate of a substance in a dialysis patient in accordance with an embodiment.

FIG. 4 is a block diagram of functional blocks, and associated input and output data, of a computer system in accordance with an embodiment.

### DETAILED DESCRIPTION OF EXAMPLE EMBODIMENTS

Embodiments of the present invention will now be described more fully hereinafter with reference to the accompanying drawings, in which some, but not all, embodiments of the invention are shown. Indeed, the invention may be embodied in many different forms and should not be construed as limited to the embodiments set forth herein; rather, these embodiments are provided so that this disclosure may satisfy applicable legal requirements. Like numbers refer to like elements throughout.

Also, it will be understood that, where possible, any of the advantages, features, functions, devices, and/or operational aspects of any of the embodiments of the present invention described and/or contemplated herein may be included in any of the other embodiments of the present invention described and/or contemplated herein, and/or vice versa. In addition, where possible, any terms expressed in the singular form herein are meant to also include the plural form and/or vice versa, unless explicitly stated otherwise. As used herein, “at least one” shall mean “one or more” and these phrases are intended to be interchangeable. Accordingly, the terms “a” and/or “an” shall mean “at least one” or “one or more”, even though the phrase “one or more” or “at least one” is also used herein. As used herein, except where the context requires otherwise owing to express language or necessary implication, the word “comprise” or variations such as “comprises” or “comprising” is used in an inclusive sense, that is, to specify the presence of the stated features but not to preclude the presence or addition of further features in various embodiments of the invention. Similarly, the expressions “as a function of” and “based on” in combination with a specified set of parameters or the like are inclusive and do not to preclude the presence or addition of further parameters.

The following description refers to “standard Kt/V”, also known as standardized Kt/V or stdKt/V, which is an established measure of dialysis adequacy. The underlying motivation for developing this measure was a need be able to compare the dialysis doses provided by different types of dialysis therapies and combinations of dialysis therapies, including both continuous and intermittent therapies. The measure was first presented by Frank Gotch in the article “The current place of urea kinetic modelling with respect to difference dialysis modalities”, published in Nephrol Dial Transplant. 13 [Suppl 6]: 10-14 (1998), which is incorporated herein by reference. Essentially, Gotch presented a method of downgrading intermittent dialyzer clearances to the equivalent of a continuous clearance (stdK) by redefining clearance as the urea generation rate divided by the average predialysis blood urea concentration. The definition assumes that the blood urea concentration is the same at the start and end of the time period t. Specifically, Gotch provided the following definition of stdKt/V:

$$stdKt / V = \frac{G \cdot t}{\overline{Cs} \cdot V} \qquad ( 1 )$$

where G is the average generation rate of urea in the dialysis patient over a predefined time period t, C̅s̅ is the average of the blood urea concentrations at the onset of the treatment sessions that are performed during the time period t, and V is the distribution volume in the patient. The time period t may include any number of treatment sessions. Traditionally, the time period t is set to a week, resulting in a “weekly stdKt/V”, but any other time period may be used, e.g. a day, 2 weeks, a month, etc. In the following, the time period t is denoted “equalization period” since the dialysis therapy is assumed to bring the patient's blood urea concentration back to the start value after the time period. Although Gotch gave the definition for urea, equation (1) is equally applicable to any other substance that is exchanged with the blood of a dialysis patient during dialysis therapy.

Over time, stdKt/V has become an established measure and is included in KDOQI—Kidney Disease Outcomes Quality Initiative, which is a broadly accepted clinical practice guideline in nephrology, see “KDOQI Clinical Practice Guideline for Hemodialysis Adequacy: 2015 Update”, Am J Kidney Dis. 2015; 66(5), pages 908-912: “Guideline 3: Measurement of Dialysis—Urea Kinetics”. The rationale behind and established use of stdKt/V is also discussed in the reference book “Replacement of Renal Function by Dialysis”, 5th revised edition, 2004, editors Hörl, Koch, Lindsay, Ronco and Winchester, Chapter 22—Adequacy of hemodialysis, pages 597-638, as well as in the article “Assessing the Adequacy of Small Solute Clearance for Various Dialysis Modalities, with Inclusion of Residual Native Kidney Function”, by Chin et al, published in Seminars in Dialysis, 30(3), 235-240 (2017).

It is important to understand that stdKt/V is not the same as Kt/V, which is an established measure that describes the effect (“dialysis dose”) of a single treatment session and is theoretically given by the logarithm of the ratio of the pre- and post-dialysis urea concentrations. More specialized equations have been developed to account for the distribution of urea in the patient, e.g. resulting in so-called single-pool Kt/V (spKt/V) or equilibrated Kt/V (eKt/V). In the following, the Kt/V for a single treatment session is denoted “session Kt/V” to be distinguished from stdKt/V.

In a clinical situation, it is difficult to calculate stdKt/V based on equation (1) given that at least G is unknown. Therefore, various algorithms for computing or estimating stdKt/V have been developed. One computation algorithm is proposed by Leypold et al. in the article “Predicting treatment dose for novel therapies using urea standard Kt/V”, published in Semin Dil 17:142-145 (2004). Here, stdKt/V is calculated from the knowledge of spKt/V for a single treatment session with no ultrafiltration (UF) or residual renal function (rrf) under the assumption that all treatment sessions during a week are equal and equally spaced. Another computation algorithm is proposed by Daugirdas et al. in the article “Standard Kt/Vurea: a method of calculation that includes effects of fluid removal and residual kidney clearance”, published in Kidney Int 77: 637-644 (2010). This algorithm accounts for UF and rrf and works well if treatment sessions are equal and evenly distributed over the week. A further computation algorithm is proposed by Leypold and Vonesh in the article “Calculating Standard Kt/V during Hemodialysis Based on Urea Mass Removed” published in Blood Purif. 8:1-7 (2018). This algorithm operates on blood urea concentration at start and end of treatment sessions and also accounts for UF. The algorithm works well if treatment sessions are equal. Yet another computation algorithm is proposed by Sternby in the article “Mathematical Representation of Standard Kt/V Including Ultrafiltration and Residual Renal Function”, published in ASAIO J. 64(5), e88-e93 (2018). This algorithm enables calculation of stdKt/V irrespective the nature, number and spacing of treatment sessions and accounts for both UF and rrf. It may also be noted that computation algorithms that do not account for rrf may be updated to add an estimated value of the contribution of rrf to the calculated value of stdKt/V, e.g. as described on pages 911-912 in the above-mentioned KDOQI Guideline. All of the foregoing publications are incorporated herein in their entirety by reference.

Common to all of these computation algorithms is that they enable calculation of stdKt/V from input data that includes either pre- and post-dialysis urea concentrations in the patient's blood for one or more treatment sessions during the equalization period t or the session Kt/V of urea for the one or more treatment sessions, as well as the duration of the respective treatment session. Certain computation algorithms also operate on further input data such as the start and end time points for the respective treatment session, the total ultrafiltration volume (UFV) removed from the blood during the during the equalization period t, and the residual renal function (rrf) of the patient, to provide a more accurate stdKt/V value. It may be noted, however, that UFV and rrf may be zero depending on therapy and patient.

In the foregoing, a distinction is made between intermittent and continuous dialysis therapies. As used herein, “continuous dialysis therapy” refers to any renal replacement therapy that is operated continuously on the patient over the equalization period t, such that the concentration of urea (or another substance) remains essentially constant in the blood of the patient. In contrast, “intermittent dialysis therapy” involves one or more renal replacement therapies each of which is operated on the patient during a respective subset of the equalization period t, causing the concentration of urea (or another substance) to vary during the equalization period t. Such renal replacement therapies may include one or more of hemodialysis, hemodiafiltration, hemofiltration, ultrafiltration and peritoneal dialysis.

Embodiments of the invention are based on the insight that the generation rate of a substance may be calculated in a simple way if stdKt/V of the substance is known, e.g. estimated by any of the above-mentioned computation algorithms, by clever use of equation (1). For example, by re-arrangement, the relative generation rate (G/V) is given by:

$$\frac{G}{V} = \frac{( stdKt / V ) \cdot \overline{Cs}}{t} \qquad ( 2 )$$

and the absolute generation rate (G) is given by:

$$G = \frac{( stdKt / V ) \cdot \overline{Cs} \cdot V}{t} \qquad ( 3 )$$

It should be noted that various assumptions may be made with respect to the computation of stdKt/V and/or the computation of C̅s̅ to simplify the calculations of the generation rate. For example, assumptions may be made to enable calculation of the generation rate based on measurements or estimations of the blood concentration of the substance at the beginning and the end of a single treatment session, even if the equalization period t involves one or more additional treatment sessions.

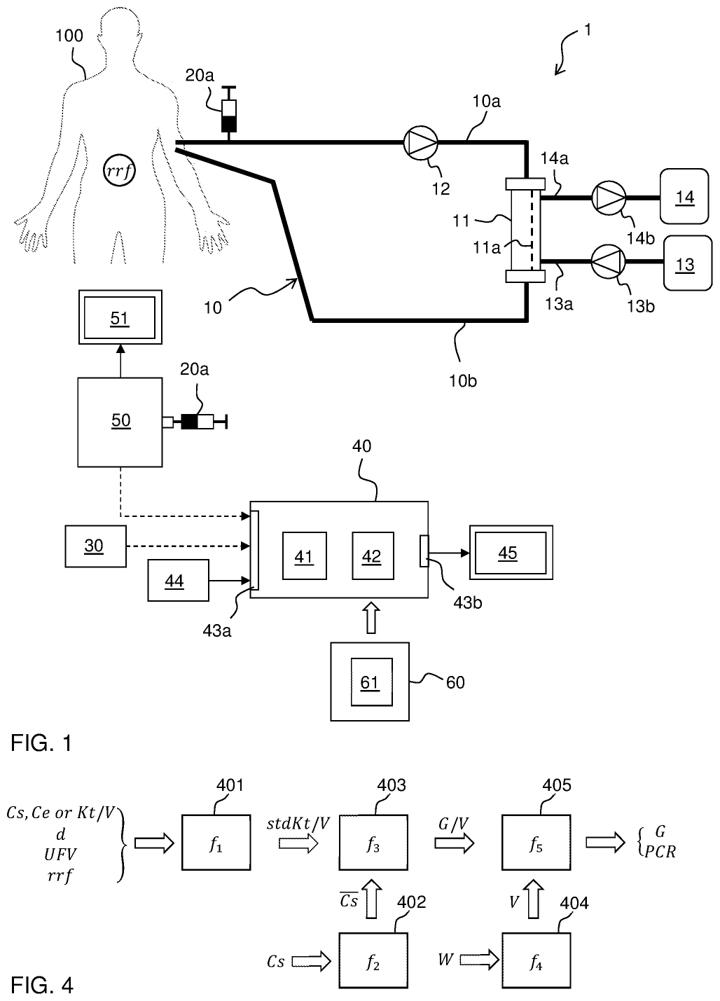

In the following, embodiments of the invention will be exemplified for measurements of urea and calculations of the relative or absolute urea generation rate, jointly designated as UGR. Reference is made to FIG. 1, which schematically depicts a blood treatment system 1 for performing hemodialysis when connected to a patient 100, i.e. a human subject. In this example, the patient 100 has a residual renal function, rrf, which is a native urea clearance by the patient's kidneys and may, e.g., be given in mL/min.

The system 1 comprises an extracorporeal blood circuit (“EC circuit”) 10 which is connected to the vascular system of the patient 100 at a blood withdrawal end and a blood return end. The connections may be performed by any conventional device, such as a needle or catheter. Blood lines or tubings are arranged to define a blood withdrawal path or limb 10a and a blood return path or limb 10b of the EC circuit 10. A blood filtration unit 11, denoted “dialyzer” herein, is connected between the withdrawal and return paths 10a, 10b. The dialyzer 11 comprises a semi-permeable membrane 11a, which is arranged to separate the dialyzer 11 into a blood compartment, which is fluidly connected to the withdrawal and return paths 10a, 10b, and a dialysis fluid compartment. A blood pump 12 is arranged in the withdrawal path 10a and is operable to draw blood from the patient 100 and pump the blood via the blood compartment of the dialyzer 11 and through the return path 10b back to the patient 100. The system 1 further comprises a source 13 of dialysis fluid. A dialysis fluid path or line 13a connects the source 13 to the dialysis fluid compartment of the dialyzer 11. Similarly, an effluent path or line 14a connects the dialysis fluid compartment of the dialyzer 11 to a sink 14 for spent dialysis fluid (also known as “effluent”). A dialysis fluid pump 13b is arranged in the dialysis fluid path 13a, and an effluent pump 14b is arranged in the effluent path 14a. The skilled person understands that the blood treatment system 1 may include further components, such as a venous drip chamber, clamps, sensors, etc.

A control device 30 is configured to generate control signals for operative components of the system 1, such as the pumps 12, 13b, 14b, to cause the system 1 to perform a treatment session in accordance with settings that have been entered into the control device 30, e.g. by a caretaker or the patient 100. The operation of a hemodialysis system 1 is known to the person skilled in the art and will not be detailed here.

FIG. 1 also illustrates a sampling device 20a, which is used for taking a sample of the patient's blood, either from the withdrawal path 10a of the EC circuit 10 (as shown) or directly from the vascular system in the patient 100. As shown, the sampling device 20a may then be connected to a blood analysis apparatus 50, which is separate from the system 1 and configured to analyze the blood sample for determination of the concentration of one or more substances, including urea. The apparatus 50 may present the result of the analysis of the respective blood sample to the operator on a display device 51. Alternatively, the blood sample may be subjected to manual laboratory analysis to yield the concentration. As known in the art, the urea concentration in blood may be given in terms of the entire urea molecule or its nitrogen-content (commonly denoted “blood urea nitrogen”, BUN).

FIG. 1 also depicts a computation device or computer system 40 which is configured to perform dedicated calculations to generate output data that allows a clinician to assess the nutritional status of the patient 100. The computer system 40, which may or may not be part of a dialysis machine, comprises a processor 41 and computer memory 42. A control program is stored in the memory 42 and executed by the processor 41 to perform the calculations. As indicated, the control program 61 may be supplied to the computer system 40 on a computer-readable medium 60, which may be a tangible (non-transitory) product (e.g. magnetic medium, optical disk, read-only memory, flash memory, etc) or a propagating signal. In the illustrated example, the computer system 40 comprises an input interface 43a for connection to one or more input devices 44 that enable an operator to supply input data, as well as an output interface 43b for connection to one or more output devices 45 for providing output data to the operator. For example, the input device(s) 44 may comprise a keyboard, keypad, computer mouse, control button, touch screen, etc, and the output device(s) 45 may comprise a display device, an indicator lamp, an alarm device, a microphone, a printer, etc.

The operator may enter input data, e.g. including the blood concentration values, into the computer system 40 via the input device 44. Alternatively or additionally, as indicated by a dashed arrow in FIG. 1, the blood analysis apparatus 50 may be connected, by wire or wirelessly, to the input interface 43a to transfer the blood concentration values to the computer system 40. Alternatively or additionally, as indicated by a dashed arrow in FIG. 1, the control device 30 may be similarly connected to transfer input data to the computer system 40. It is also conceivable that the computer system 40 is integrated in the control device 30, or vice versa.

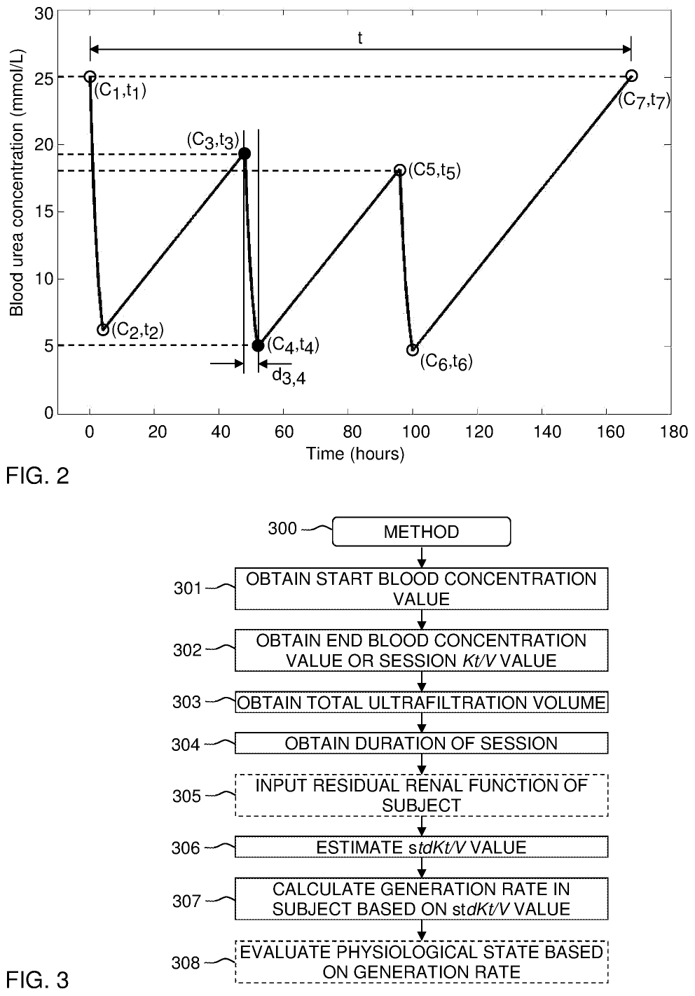

FIG. 2 shows an example of the urea concentration in the blood of the patient 100 over a time period of a week, in which the patient is subjected to intermittent dialysis in three separate treatment sessions. In each treatment session, the EC circuit 10 is connected to the vascular system of the patient 100, as shown in FIG. 1, whereupon the system 1 is operated to perform a blood treatment procedure in which uremic solutes and water are removed from the patient's blood via the dialyzer membrane 11a. Such solutes include, without limitation, urea, creatinine, beta-2-microglobin (B2M), beta-trace protein, Vitamin B12, etc.

In FIG. 2, the treatment sessions are performed between time points t1-t2, t3-t4 and t5-t6, respectively, each resulting in a significant reduction in the blood urea concentration, from C1 to C2 in the first session, from C3 to C4 in the second session, and from C5 to C6 in the third session. Between the treatment sessions, the blood urea concentration rises as a result of metabolic processes in the patient, from C2 to C3 between the first and second sessions, from C4 to C5 between the second and third sessions, and from C6 to C7 after the third session until time point t7. It may be noted that the residual renal function (rrf) is zero in this example. FIG. 2 is characteristic of dialysis therapy performed three times each week, e.g. Monday, Wednesday and Friday. As seen, the blood urea concentration is approximately the same at the beginning of the week and the end of the week. In fact, all dialysis therapies may be sub-divided into time periods that start and end at approximately the same blood urea concentration.

Commonly, the time period is one or more days or one or more weeks. This time period is thus the equalization period t in the definition of stdKt/V. FIG. 2 also illustrates that the predialysis blood urea concentration C1, C3, C5 decreases monotonically from the first to the last session within the equalization period. This is a characteristic of dialysis therapies that involve two or more temporally spaced treatment sessions during the equalization period, in which at least one temporal spacing differs from other temporal spacing(s).

FIG. 3 illustrates a method 300 for determining UGR in a dialysis patient in accordance with an embodiment. The method 300 may be performed by the computer system 40 in FIG. 1 and will be exemplified with reference to FIG. 2. The illustrated embodiment is based on the understanding that it is often possible, e.g. by use of the above-mentioned computation algorithms, to estimate stdKt/V with sufficient accuracy for a single selected treatment session even if the equalization period t includes further treatment session(s). The selected session is preferably a mid-session among the sessions in the equalization period, e.g. the second session among three sessions, the second or third session among four sessions or the third session among five sessions. As understood from the foregoing, some of the available computation algorithms operate on the session Kt/V, which may be measured by a dedicated measurement device or be computed based on pre- and postdialysis concentrations. For example, the above-mentioned spKt/V or eKt/V may form the session Kt/V that is input to the computation algorithm. Other computation algorithms are configured to operate on the measured pre- and postdialysis concentrations instead of the session Kt/V. Thus, the method 300 may differ depending on the type of input data.

The method 300 comprises steps 301-305 of obtaining input data for calculation steps 306-307, which are followed by an optional evaluation step 308. Step 301 obtains a first measured value representative of the blood urea concentration at the start of the selected session. Step 302 obtains either a second measured value representative of the blood urea concentration at the end of the selected session, or the session Kt/V of urea for the selected session. In the example of FIG. 2, the first and second measured values may be the concentration values C3 and C4 at time points t3, t4 of the second session, indicated by filled dots. In FIG. 1, the first and second measured values may be entered by the caretaker via the input device 44 or be transferred electronically from the blood analysis apparatus 50. The session Kt/V may also be entered by the caretaker via the input device 44 and may be given by any suitable measurement device (not shown) in the dialysis system 1, including but not limited to the devices discussed in the Summary section. Alternatively, the caretaker may input an in-vivo urea clearance K, e.g. given by such a measurement device, whereupon step 302 computes the session Kt/V in conventional manner The session Kt/V or urea clearance K may alternatively be electronically transferred from the measurement device or the control device 30 to the computer system 40. In a further alternative, the session Kt/V is computed separately based on the pre- and postdialysis concentrations, e.g. in accordance with any established equation, and is then input to the computer system 40 in step 32.

Step 303 obtains the total ultrafiltration volume, UFV, for the treatment sessions during the equalization period. The total UFV is generally known to the caretaker. In FIG. 1, the total UFV may be entered by the caretaker via the input device 44 or transferred electronically from the control device 30. The total UFV may be input as an aggregated value for the equalization period or as an UFV value for the respective session. If the UFV is known to be zero for a specific therapy, step 303 may be omitted.

Step 304 obtains the duration of the selected session. In FIG. 1, the duration may be entered by the caretaker via the input device 44 or transferred electronically from the control device 30. In FIG. 2, the duration of the second session is d3,4.

If the patient has a residual renal function, rrf, step 305 may be included to obtain data representative of the rrf, e.g. quantified as a urea clearance value K_{rrf}. The rrf data may be entered by the caretaker via the input device 44.

Step 306 operates on the input data from steps 301-305 to compute an estimated value of stdKt/V for urea, e.g. by use of any of the above-mentioned computation algorithms In one example, stdKt/V is computed as a function of the first and second measured values, the duration of the selected session, the total UFV. In another example, the stdKt/V is computed as a function of the session Kt/V, the duration of the selected session and the total UFV. It is generally recognized that a more accurate value of stdKt/V may be obtained by accounting for recirculation and rebound in the patient when determining the post-dialysis concentration, i.e. the second measured value. This type of value is known as “equilibrated concentration” in the art. The equilibrated concentration may be obtained by waiting 30 minutes after the end of the treatment session before obtaining the postdialysis blood sample, or by mathematically manipulating the concentration value of a blood sample taken at the actual end of the session. Thus, if the second measured value is the concentration C4 at t4 (FIG. 2), step 306 may convert the second measured value to an equilibrated urea concentration as part of the stdKt/V calculation. Similarly, step 306 may convert the session Kt/V obtained by step 302, e.g. spKt/V, into an equilibrated session Kt/V (eKt/V). In other embodiments, the equilibrated concentration or eKt/V may be obtained by step 302 and input in step 306.

Step 307 computes the UGR by use of equation (2) or (3) above, based on stdKt/V, t and C̅s̅. Here, t is the known length of the equalization period. The average pre-dialysis blood urea concentration C̅s̅ may be estimated, preferably as a function of the first measured value. From FIG. 2, it is realized that among the pre-dialysis concentration values C1, C3 and C5, the value C3 lies closest to the true C̅s̅ (which is the average of C1, C3 and C5). Thus, the estimated value of C̅s̅ may be given by C3 or set in relation to C3, e.g. by multiplication with a predetermined correction factor and/or addition of a predetermined correction value (positive or negative). If equation (3) is used, the distribution volume V in the patient 100 may be obtained in a preceding step (not shown). For example, the distribution volume V may be known to and input by the caretaker via the input device 44 or electronically transferred from the control device 30. For urea (and creatinine), the distribution volume V may be approximated by the total body water (TBW), which may be estimated for the patient. For example, the caretaker may input the dry weight or body weight of the patient, and possibly further patient data such as sex, age, height, etc, thereby allowing the method 300 to estimate TBW of the patient 100, e.g. by assuming that TBW is a given percentage of the body weight of the patient, or by using any established formula such as the Watson formula, the Hume-Weyers formula or the Chertow formula. Alternatively, TBW may be measured on the patient, e.g. by bioelectrical impedance analysis (BIA).

Subsequent to step 307, the UGR may be output, e.g. for presentation on the display 45, and/or stored in memory 42 in association with a patient ID for the patient 100. As indicated in FIG. 3, the method 300 may also involve a step 308 of evaluating the UGR for assessment of the physiological status of the patient 100, e.g. the nutritional status, and displaying a parameter value representing the physiological status. For example, the physiological status may be evaluated by comparison to previously computed UGR values for the patient and/or by comparing the current UGR value to a threshold. It is also conceivable that the net protein catabolic rate (PCR), also known as the protein equivalent of nitrogen appearance (PNA), is computed from the UGR and presented to the caretaker.

The selection of computation algorithm for use in step 306 may be a trade-off between desired accuracy, computational complexity and availability of input data. A less complex computation algorithm may be selected if the dialysis therapy reasonably satisfies the underlying assumption of the computation algorithm, e.g. if the sessions may be considered equal and equally spaced during the equalization period. However, as an alternative to operating on blood urea concentrations or session Kt/V for only one session among plural sessions, it is conceivable that step 306 operates on corresponding data for further or all sessions during the equalization period. In the example of FIG. 2, the method 300 may involve obtaining the session Kt/V for each session (or the concentration values C1-C7) and the corresponding time points tl-t7 and applying one of the known computation algorithms to obtain a substantially exact stdKt/V value. Further, C̅s̅ may be computed as the average of any combination of C1, C3 and C5 and used with equation (2) or (3).

FIG. 4 is a block diagram of an arrangement of computation modules or units 401-405 that are configured to jointly compute an UGR parameter based on input data, e.g. in accordance with the method 300. The arrangement may be included in the computer system 40 in FIG. 1, and the respective module may be implemented by a combination of software instructions and hardware components (including processor 41 and memory 42), or exclusively by hardware components. Module 401 operates a predefined function f₁ on first input data to generate a stdKt/V value. In the illustrated example, the first input data comprises either first and second measured values Cs, Ce, i.e. start and end blood urea concentrations for a treatment session, or a session Kt/V value for the treatment session, and further comprises the duration d of the treatment session, the total UFV for the equalization period t and rrf, if present. Module 402 operates a predefined function f₂ on second input data comprising the first measured value C̅s̅ to estimate the average predialysis blood urea concentration Cs. Module 403 operates a predefined function f₃ on the stdKt/V value and C̅s̅ to compute a relative urea generation rate G/V. Module 404 operates a predefined function f₄ on third input data comprising the weight W of the patient to estimate the distribution volume V. Module 405 operates a predefined function f₅ on the G/V value and the distribution volume V to compute the absolute urea generation rate G or a PCR value.

As noted above, all of the foregoing embodiments are equally applicable to any other substance that is removed from the patient's blood in dialysis therapy. Thus, by obtaining the session Kt/V for any such substance, or the first and second measured values for the substance, the foregoing methodology provides the absolute or relative generation rate of the substance in the patient. For example, the substance may be creatinine or B2M, as discussed in the Background section. In one embodiment, the generation rate for two or more substances are computed and jointly evaluated for assessment of the physiological status of the patient (cf. step 308 in FIG. 3). For example, the generation rate of urea and creatinine may be jointly evaluated for assessment of nutritional status.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and the scope of the appended claims.

Further, while operations are depicted in the drawings in a particular order, this should not be understood as requiring that such operations be performed in the particular order shown or in sequential order, or that all illustrated operations be performed, to achieve desirable results. In certain circumstances, parallel processing may be advantageous.

1-18. (canceled)

19. A computer-implemented method of calculating a generation rate of a substance in a dialysis patient, said method comprising:
electronically obtaining a first concentration value for the substance in the blood of the dialysis patient at the start of a treatment session of intermittent dialysis therapy;
electronically obtaining a standard Kt/V value for the substance over a predefined time period that includes the treatment session; and
electronically calculating the generation rate of the substance in the dialysis patient as a function of the standard Kt/V value, the first concentration value and the predefined time period.

20. The method of claim 19, further comprising:
electronically obtaining a session Kt/V value for the treatment session or a second concentration value for the substance in the blood of the dialysis patient at the end of the treatment session;
electronically obtaining a volume value representative of total fluid volume removed from the blood during the predefined time period; and
electronically obtaining a duration of the treatment session,
wherein said electronically obtaining the standard Kt/V value comprises electronically computing the standard Kt/V value as a function of the volume value, the duration, and one of the session Kt/V value or the first and second concentration values.

21. The method of claim 19, wherein the substance is urea.

22. The method of claim 19, wherein the substance is creatinine.

23. The method of claim 19, wherein the substance is beta-2-microglobulin.

24. The method of claim 19, wherein said calculating the generation rate comprises multiplying the standard Kt/V value, the reciprocal of the predefined time period, and an estimated concentration value, which is representative of an average predialysis concentration of the substance in the blood of the dialysis patient during the predefined time period.

25. The method of claim 24, further comprising determining the estimated concentration value as a function of the first concentration value.

26. The method of claim 24, further comprising setting the estimated concentration value in relation to the first concentration value.

27. The method of claim 24, further comprising: computing the estimated concentration value as an average of the first concentration value and one or more further concentration values for the substance in the blood of the dialysis patient at the start of one or more further treatment sessions of intermittent dialysis therapy during the predefined time period.

28. The method of claim 19, wherein the predefined time period is selected so that the concentration of the substance in the blood of the dialysis patient is substantially equal at the start and end of the predefined time period.

29. The method of claim 19, wherein the predefined time period is a week.

30. The method of claim 19, wherein the predefined time period includes one or more further treatment sessions, and wherein the standard Kt/V value is estimated to include the one or more further treatment sessions.

31. The method of claim 30, wherein the standard Kt/V value is estimated in absence of concentration values for the substance in the blood of the dialysis patient during the one or more further treatment sessions and in absence of a Kt/V value for the one or more further treatment sessions.

32. The method of claim 31, wherein the first concentration value of said treatment session is closest to an average predialysis concentration of the substance in the blood of the dialysis patient during the predefined time period, compared to an expected concentration value for the substance in the blood of the dialysis patient at the start of the respective further treatment session.

33. The method of claim 19, further comprising one or more of:
displaying the generation rate, evaluating the generation rate for assessment of a physiological status of the dialysis patient, or displaying a parameter value representing the physiological status of the dialysis patient.

34. The method of claim 19, which is performed subsequent to said treatment session.

35. The method of claim 19, further comprising generating an indicator indicative of a physiological status of the patient based on the calculated generation rate.

36. A non-transitory, computer-readable medium storing instructions, which when executed by a processor, cause the processor to:
receive a first concentration value for a substance in the blood of the dialysis patient at the start of a treatment session of an intermittent dialysis therapy;
receive a standard Kt/V value for the substance over a predefined time period that includes the treatment session; and
calculate the generation rate of the substance in the dialysis patient as a function of the standard Kt/V value, the first concentration value and the predefined time period.

37. A system for calculating a generation rate of a substance in a dialysis patient comprising:
a diaysis mahine including:
a memory; and
a processor in communication with the memory, the processor configured to:
receive a first concentration value for the substance in the blood of the dialysis patient at the start of a treatment session of an intermittent dialysis therapy performed by the dialysis machine,
receive a standard Kt/V value for the substance over a predefined time period that includes the treatment session, and
calculate the generation rate of the substance in the dialysis patient as a function of the standard Kt/V value, the first concentration value and the predefined time period.
